(19) United States
(12) Patent Application Publication
(10) Pub. No.: US 2008/0014163 A1
(43) Pub. Date: Jan. 17, 2008

(54) Protective Applicator, Composition, and Method

(76) Inventors: Stacey Leigh Grabiner, Westfield, NJ (US); Dakota Reid Smith, New York, NY (US); Robert Walter Sandewicz, Monroe Township, NJ (US); James Carl Paccagnini, Fairfield, NJ (US)

Correspondence Address: JULIE BLACKBURN;REVLON CONSUMER PRODUCTS CORPORATION, 237 PARK AVENUE, NEW YORK, NY, 10017, US

(21) Appl. No.: 11/457,476

(22) Filed: Jul. 14, 2006

(51) Int. Cl. A61K 8/81

(52) U.S. Cl. 424/70.7; 132/202

(57) ABSTRACT

A product for protecting a non-application keratinous surface from inadvertent application of cosmetic being applied to an application keratinous surface comprising a protective composition contained in a receptacle having an applicator for applying the protective composition to the area to be protected prior to application of the cosmetic and method for protecting a non-application keratinous surface from inadvertent application of cosmetic being applied to an application keratinous surface comprising applying a protective composition to the non-application keratinous surface prior to applying cosmetic to the application keratinous surface.

### TECHNICAL FIELD

The invention is in the field of applicators that dispense compositions that are used to protect keratinous surfaces during the application of makeup so that the makeup applied does not stray onto areas where it is not intended to be applied.

### BACKGROUND OF THE INVENTION

One common problem with makeup application is that when makeup is applied to the desired area of the keratinous surface it may sometimes stray to another area where it is not wanted. This means that the consumer must remove the cosmetic from the undesired areas. Examples of stray cosmetic application include nail enamel accidentally applied to the cuticle area, or hair color that strays onto facial skin surrounding the hair line, and so on. One of the most frustrating problems is for consumers that are at-home nail enamel users. It is relatively easy to neatly apply nail enamel to the nails of the non-dominant hand using the dominant hand. However, when the consumer attempts to apply nail enamel to the nails of the dominant hand using the non-dominant hand to wield the brush, the application is often difficult and messy, with stray nail enamel sometimes ending up on the cuticle area.

Women who can afford to go to salons combat the stray nail enamel problem by having nail technicians perform their manicures. A nail technician applies the enamel to the nail surface and uses her nails or a wooden pusher tool to remove the excess nail enamel from the cuticle area.

There is a need for products that protect keratinous surfaces from stray cosmetic during the cosmetic application process, particularly in the nail enamel and hair dye field. A most desired product would be one where the protective product is applied to the area desired to be protected and, instead of being removed after the cosmetic is applied, could be left on the skin.

It is an object of the invention to provide a product comprised of a protective composition operable to protect a keratinous surface from stray cosmetic during the cosmetic application process where such product can be left on the keratinous surface after the cosmetic application process has been completed.

It is an object of the invention to provide a packaged protective composition comprised of a receptacle for containing the composition, said receptacle having an applicator designed to dispense the protective composition in exactly the place to be protected, and wherein the protective composition is left on the keratinous surface after cosmetic application.

It is an object of the invention to provide a method for protecting a keratinous surface from stray cosmetic during the cosmetic application process by applying a composition to the surface desired to be protected, then applying the cosmetic to the desired area.

It is a further object of the invention to provide a receptacle comprised of a tube having contained therein a protective cosmetic composition, and an applicator suitable for applying the protective composition to the keratinous surface desired to be protected.

### SUMMARY OF THE INVENTION

The invention is directed to a product for protecting a non-application keratinous surface from inadvertent application of cosmetic being applied to an application keratinous surface comprising a protective composition contained in a receptacle having an applicator for applying the protective composition to the area to be protected prior to application of the cosmetic.

The invention is also directed to a method for protecting a non-application keratinous surface from inadvertent application of cosmetic being applied to an application keratinous surface comprising applying a protective composition to the non-application keratinous surface prior to applying cosmetic to the application keratinous surface.

### DETAILED DESCRIPTION

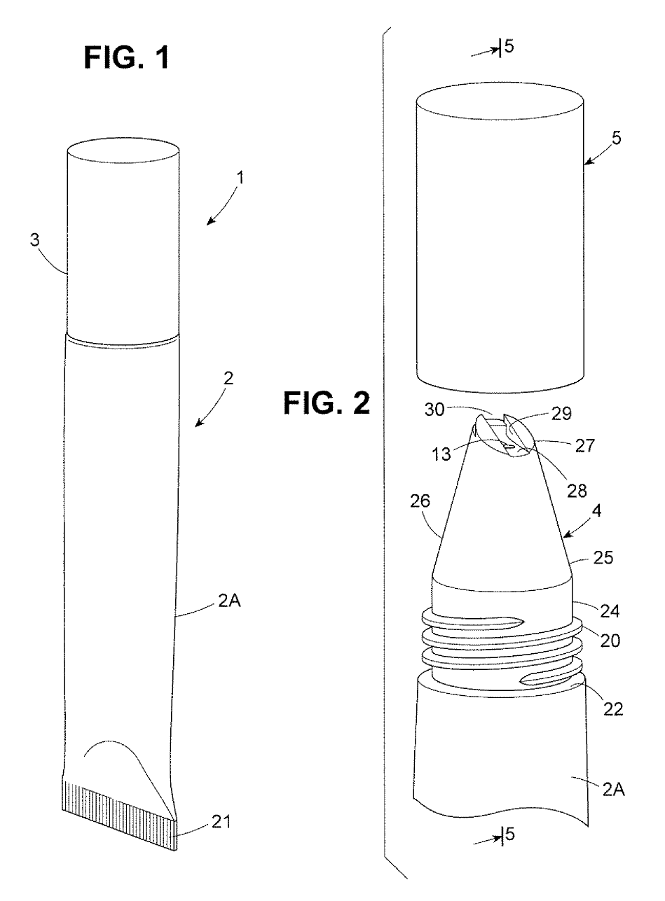

FIG. 1: depicts one type of receptacle in accordance with the invention comprised of a tube with cap in the closed position.

FIG. 2: is an exploded view of the applicator portion of the receptacle with the cap removed, showing the tip configuration.

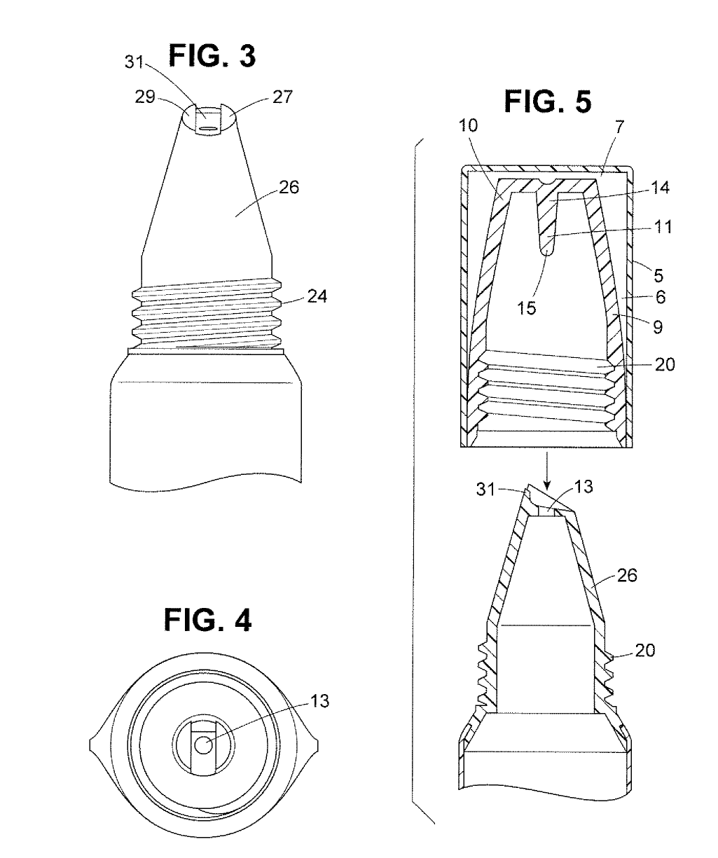

FIG. 3: depicts the applicator showing the configuration of the tip.

FIG. 4: is a top plan view of the applicator of FIG. 3.

FIG. 5: is a cutaway view taken across 5-5 of FIG. 1, showing the internal configuration of the applicator and cap.

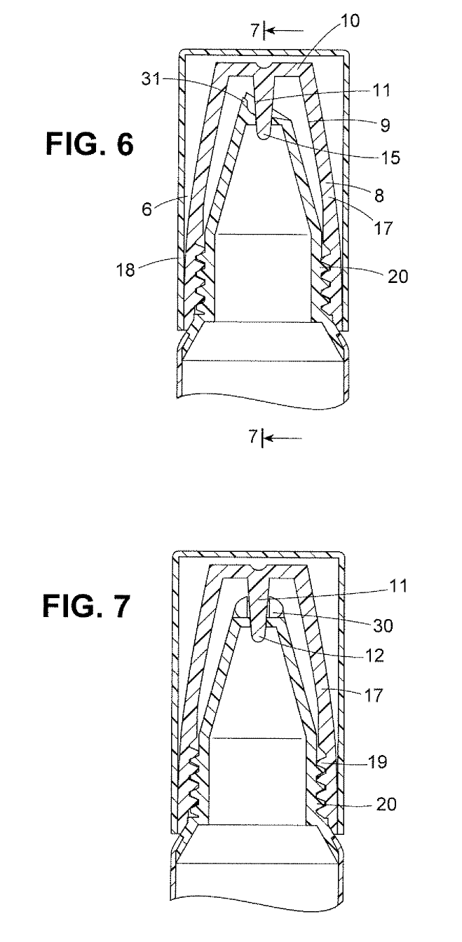

FIG. 6: is a cross sectional view of the applicator and cap in the closed position.

FIG. 7: is a cross sectional view of the applicator and cap taken across 7-7 of FIG. 6.

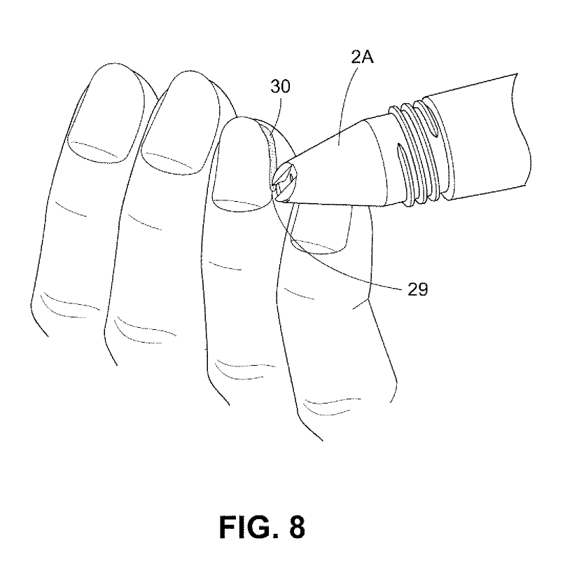

FIG. 8: shows how the product is applied to a non-application surface such as nail cuticles.

### DETAILED DESCRIPTION OF THE DRAWINGS

### I. Definitions

A. The term “application keratinous surface” means a keratinous surface where cosmetic is intended to be applied. For example, in the case where the cosmetic is nail enamel the application keratinous surface is the nail. In the case where the cosmetic is hair color, the application keratinous surface is the skin surrounding the hair line.

B. The term “non-application keratinous surface” means a keratinous surface surrounding the application keratinous surface where, during application of cosmetic to the application keratinous surface, it sometimes happens that stray cosmetic is inadvertently applied to the non-application keratinous surfaces.

C. The term “protective composition” means a composition that is applied to the non-application keratinous surface in order to protect that surface from inadvertent application of cosmetic that is being applied to an application keratinous surface.

### II. The Product

FIG. 1 depicts the product 1 of the invention. The product 1 comprises a receptacle 2 such as a tube 2A for containing a protective composition (not shown). The receptacle 2 preferably has a closure 3 and applicator 4. Applicator 4 may be molded to tube 2A or it may be a separate piece that is affixed to tube 2A.

A. The Closure

The closure 3 is preferably in the form of a cylindrical cap 5. Preferably cylindrical cap 5 has sidewalls 6 and top wall 7. Preferred is where cylindrical cap 5 contains an insert 8 that fits on the inside of cylindrical cap 5. Insert 8 has side walls 9 that slope upwardly and merge into a insert cap 10 that contains a downward tine 11 of a size and shape that is designed to fit into orifice (to be later described) and serve as a plug 12 for orifice 13 while cylindrical cap 5 is affixed to the receptacle 2 when the tube 2A is in the closed position. Tine 11 preferably has a tooth-like configuration with a wider base portion 14 and a narrower distal portion 15 that is shaped to fit into orifice 13 and serve as a plug 12 when the cylindrical cap 5 is in place.

When cylindrical cap 5 is removed as depicted in FIGS. 2 and 5, tine 11 and plug 12 are removed from receptacle 2 and orifice 13 respectively so that the composition (to be later described) can be extruded from the orifice 13. When cylindrical cap 5 is affixed to receptacle 2 the tine 11 serving as a plug 12 ensures that the orifice does not become plugged with composition that could otherwise dry and form a blockage in the orifice 13 such that the user cannot squeeze composition from the receptacle 2.

Preferably sidewalls 9 of insert 8 converge slightly from midpoint 17 as they slope upwardly to terminate in insert cap 10, leaving an open space 16 between side walls 6 of cylindrical cap 5 and side walls 9 of insert 8. Similarly, sidewalls 9 of insert 8 diverge slightly from the midpoint 17 such that they abut inner 18 sidewalls 6 of cylindrical cap 5. Sidewalls 9 of insert 8 preferably contain threads 19 that mate with threads 20 of applicator 4 when cylindrical cap 5 is affixed to receptacle 2.

Cylindrical cap 5 and insert 8 may be made of a variety of thermoplastic materials including polyethylene, polypropylene, ABS, and so on. The type of material is not limited provided that it is compatible with the protective composition that is stored in the receptacle 2.

B. The Receptacle

The receptacle 2 is preferably a tube 2A. Tube 2A has a crimped end 21. It is preferred that the tube 2A be crimped after it is filled with the composition. This is referred to as a “bottom fill” in the cosmetics industry. Tube 2A may be made of a variety of thermoplastic materials similar to those mentioned above with respect to the closure. The tube 2A is preferably made of a thermoplastic material that is compressible such that when the user squeezes the tube 2A with the fingers the composition is extruded from orifice 13. The tube 21 may have a neck 22, if desired.

While a tube 2A is one embodiment of the type of receptacle 2 that may be used, the receptacle 2 may be in a variety of other forms such as packettes, or tottles, and the like.

C. The Applicator

Affixed to the tube 2 is an applicator 4. Applicator 4 comprises a base portion 23 that is connected or affixed to the tube upper end 24. Applicator base portion 23 preferably contains threads 20 molded thereon that are designed to mate with corresponding threads 18 on the inner surface of cylindrical cap 5.

The applicator has a top portion 25 that forms an inverted cone 26 which narrows to form a tip 27 which contains a channel 28 that is formed between two walls 29 that have straight side edges 30. Channel 28 has a slightly upcurving edge 31 that has a height of about one half of the height of straight side edge 30 of wall 29. The upcurving edge 31 and straight side edges 30 of walls 29 form an area that molds the shape of the composition that ix extruded from the tube 2A such that it is the optimal diameter for applying to the non-application keratinous surface.

Top portion 25 of applicator 4 may be in a variety of shapes. However, the inverted cone 26 shape is most preferred because it causes the composition that is extruded from the tube 2A to slowly converge as it moves toward the tip 27 and is extruded out of the orifice. When composition is extruded from orifice 13 the shape of channel 28 dictates the shape of the stream of composition that is extruded. In this case, the composition is extruded in a ribbon like shape that can easily be applied to a non-application area such as nail cuticles 29 as best depicted in FIG. 8. As is seen in FIG. 8, the composition is applied to the nail cuticle 29 in a ribbon like form that is extruded from the tip 27 of applicator 4 as the user squeezes the tube 2A.

D. The Protective Composition

The protective composition may be in the form of a liquid, solid, or semi-solid. Preferably it is in the form of a semi-solid or solid composition. Most preferred is where the protective composition is a viscous semi-solid that exhibits a rheology that is similar to that of peanut butter or butter.

The protective composition may be made of organic materials or silicones. Examples of suitable organic materials include oils and waxes combined in such an amount as to provide a semi-solid composition. Suitable waxes or wax-like materials include animal, vegetable, mineral, synthetic, or silicone waxes such as petrolatum, candelilla wax, carnauba wax, polyethylene, ceresin; alkylated dimethicones such as stearyl dimethicone, behenyl dimethicone, and so on. Suitable amounts of wax may range from about 15-100%, preferably from about 25-99%, more preferably from about 45-95% by weight of the total composition.

The wax or wax like materials may be used alone or combined with various oils to slightly modify the rheology. Suitable oils include animal, vegetable, mineral, synthetic, or silicone oils. If present such oils may range from about 0.001-45%, preferably from about 0.005-40%, more preferably from about 0.05-35% by weight of the total composition. Examples of oils include liquid paraffin; dimethicone; phenyl substituted silicones; mono-, di- or triesters such as isopropyl palmitate, isopropyl myristate, diisostearyl malate, trioctyldodecyl citrate, triisostearyl citrate, and so on.

The protective composition may also contain other ingredients such as preservatives, pigments, surfactants, humectants, and the like.

The largest benefit of the protective composition 30 is that it protects the non-application surface during the time the makeup is applied to the application surface. When the makeup application is completed, the protective composition 30 can either be removed from the non-application surface or massaged into the keratinous surface where it will serve as a moisturizer and humectant for the non-application keratinous surface.

### II. The Method

The invention is also directed to a method for protecting a non-application keratinous surface from inadvertent application of cosmetic composition during the application of a cosmetic composition to an application keratinous surface comprising applying to the keratinous surface to be protected a protective composition that may be used to otherwise treat or condition the keratinous surface.

In the method of the invention the user extrudes the protective composition 30 from the tube 2A orifice 13 onto a non-application keratinous surface such as nail cuticles 29. The protective composition 30 serves as a barrier such that when the cosmetic is applied to the application surface, if any stray amounts are inadvertently applied to the non-application surface the barrier formed by the protective composition 30 will prevent the inadvertently applied cosmetic from staining or becoming affixed to the keratinous surface.

In the case where the cosmetic composition applied to the application surface is nail enamel, the nail enamel may inadvertently be applied to the non-application surface which is the surrounding cuticles. If the protective composition 30 is applied to the cuticle area, or non-application surface, prior to nail enamel application, any stray nail enamel that is applied to the non-application surface will not stain the cuticles because the barrier formed by the protective composition.

The protective composition can be tissued off once the nail enamel has dried or it may simply be massaged into the cuticles and hands.

The invention will be further described in connection with the following examples which are set forth for the purposes of illustration only.

### EXAMPLE 1

A protective composition was prepared as follows:

| Ingredient | w/w % |
|---|---|
| Petrolatum | 93.2 |
| Trioctyldodecyl citrate | 5.0 |
| Mineral oil, coconut oil, aloe extract | 0.1 |
| Hexanediol, caprylyl glycol | 0.70 |
| Pigment grind (in trioctyldodecyl citrate) | 0.60 |

The composition was a viscous semi-solid suitable for use as a protective composition for application to cuticles prior to nail enamel application or to the skin surrounding the hairline prior to application of hair dye.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A product for protecting a non-application keratinous surface from inadvertent application of cosmetic being applied to an application keratinous surface comprising a protective composition contained in a receptacle having an applicator for applying the protective composition to the area to be protected prior to application of the cosmetic.

2. The product of claim 1 wherein the protective composition is a viscous semi-solid composition.

3. The product of claim 2 wherein the non-application keratinous surface is the nail cuticle.

4. The product of claim 3 wherein the receptacle comprises a tube, a closure, and an applicator.

5. The product of claim 4 wherein the applicator comprises a base portion, a top portion forming an inverted cone.

6. The product of claim 5 wherein the top portion forming an inverted cone contains an orifice in a channel that is formed between two walls that have straight side edges.

7. The product of claim 6 wherein channel has a slight upcurving edge.

8. The product of claim 7 wherein the straight side edges and upcurving edge form a area that molds the same of the protective composition that is extruded from the receptacle.

9. The product of claim 8 wherein the tube has a crimped end.

10. The product of claim 8 wherein the closure is in the form of a cylindrical cap.

11. The product of claim 10 wherein cylindrical cap contains an insert having sidewalls that slope upwardly and merge into an insert cap.

12. The product of claim 11 wherein insert cap contains a downward tine that fits into the orifice in the applicator and forms a plug.

13. The product of claim 12 wherein the protective composition is a semi-solid viscous composition.

14. A method for protecting a non-application keratinous surface from inadvertent application of cosmetic being applied to an application keratinous surface comprising applying a protective composition to the non-application keratinous surface prior to applying cosmetic to the application keratinous surface.

15. The method of claim 14 wherein the non-application keratinous surface is the nail cuticles or the skin surrounding the hair line.

16. The method of claim 14 wherein the protective composition is a viscous semi-solid composition.

17. The method of claim 16 wherein the protective composition is applied to the non-application keratinous surface using a product comprised of a receptacle having an applicator with a top portion in an inverted cone shape and an orifice in a channel that is formed between two walls that have straight side edges and a slight upcurving edge.

18. The method of claim 17 wherein the receptacle is a squeezable tube.

19. The method of claim 17 wherein the product contains a closure in the form of a cylindrical cap having an insert with side walls and an insert cap.

20. The method of claim 19 wherein the insert cap has a downward tine that fits into orifice and serves as a plug.
